(19) United States
(12) Patent Application Publication
(10) Pub. No.: US 2017/0246223 A1
(43) Pub. Date: Aug. 31, 2017

(54) Combination Therapy for Solid Tumour Cancer

(71) Applicant: UNIVERSITY COLLEGE CORK, NATIONAL UNIVERSITY OF IRELAND, CORK, Cork (IE)

(72) Inventors: Mark Tangney, Glanmire (IE); Panos Lehouritis, Athens (GR)

(21) Appl. No.: 15/442,043

(22) Filed: Feb. 24, 2017

Related U.S. Application Data
(60) Provisional application No. 62/299,998, filed on Feb. 25, 2016.

(51) Int. Cl. A61K 35/741; A61K 9/00; A61K 35/744; A61K 31/7076; A61K 35/745; A61K 31/7068; A61K 31/136; A61K 31/7048; A61K 35/747

(52) U.S. Cl. CPC A61K 35/741; A61K 31/7048; A61K 9/0019; A61K 35/744; A61K 35/747; A61K 35/745; A61K 31/7068; A61K 31/136; A61K 31/7076; A61K 2035/11

(57) ABSTRACT

A combination comprising an effective dose of a chemotherapeutic-activating wild-type bacterium and an effective dose of a chemotherapeutic agent that is responsive to the chemotherapeutic activating wild-type bacterium, for use in a method of preventing or treating a solid tumour cancer in a mammal.

### RELATED APPLICATION(S)

This application claims the benefit of U.S. Provisional Application No. 62/299,998, filed on Feb. 25, 2016. The entire teachings of the above application are incorporated herein by reference.

### BACKGROUND

Tumour responses to chemotherapy vary, and deeper insight into the reasons for therapeutic failure with certain tumours while other apparently similar tumours respond well, stands to guide and improve existing therapeutic regimes, while informing the development of new treatments. Bacteria have been linked with various cancers in a number of ways. For example, bacterial-induced inflammation has been linked with cancer promotion and progression, via indirect distal effects from the GIT microbiome, or directly such as in the case of Helicobacter pylori. Recent research in experimental tumours revealed that gut bacteria may influence the outcome of chemotherapy indirectly via influencing the immune system. For decades, naturally occurring bacteria of different types have been isolated from patient tumours of various histological types. In parallel to this, it is well known that deliberate systemic administration of bacteria to animals or patients results in selective replication within solid tumours. The reasons for tumour tissue targeting by bacteria and their proliferation within them believed to be multifactorial, with a number of key features distinguishing the tumour phenotype from normal tissues: ‘Leaky’ tumour vasculature may provide bacterial access to tissue; the immune-suppressed environment of the tumour protects bacteria from immune surveillance; the low oxygen potential of necrotic and hypoxic regions of tumours facilitates anaerobic bacterial growth (unlike other tissues); tumour necrosis provides nutrients favourable to bacterial growth. The bacterial populations naturally present within malignant and non-malignant tissue of the breast, and the presence of a range of bacteria in the breast tissue of cancer patients, has recently been reported.

Viaud et al [1] have reported that the intestinal microbiota can have an indirect impact on the effects of drugs, in the context of the effects of gut bacteria on the immune system, and can impact the treatment effect of such immune-targeted drugs (cylophosphamide).

### SUMMARY OF THE INVENTION

The present invention is based on the finding that certain wild-type bacteria can have an effect on certain chemotherapy drugs. The effect can be positive, where the cytotoxic effect of the drug on a tumour is enhanced in the presence of a specific wild-type bacteria, or negative, where the cytotoxic effect is inhibited in the presence of the specific wild-type bacteria. Table 1 shows the effect of a two wild-type bacteria (E. coli and Listeria welshimeri) on the cytotoxic effect of 29 conventional chemotherapeutic drugs in vitro, in which 5 have improved cytotoxic effect, 10 have a reduced cytotoxic effect, and 14 are unaffected, relative to a reference cytotoxic effect. FIG. 8 shows the effect of a range of wild-type bacteria on the cytotoxic effect of a number of enzyme-modifiable chemotherapeutic drugs.

The invention provides novel tailored therapies for cancer in individuals that is informed by (i) a synergistic combination of a specific wild-type bacterium and a chemotherapeutic drug (or drugs) that responds to the specific wild-type bacterium, or (ii) a combination of a chemotherapeutic drug (or drugs) and an anti-bacterial agent, in which the anti-bacterial agent is directed against a wild-type bacterium known to inhibit the chemotherapeutic effect of the chemotherapeutic drug or drugs.

In a first aspect, the invention provides a method for identifying a chemotherapeutic agent that is responsive to a specific wild-type bacterium (hereafter “wild-type bacterium-responsive chemotherapeutic agent”) comprising the steps of incubating the specific wild-type bacterium with a candidate chemotherapeutic agent, separating the candidate chemotherapeutic agent from the wild-type bacterium, incubating the candidate chemotherapeutic agent with a cancer cell, and determining whether there is an increase in a cytotoxic effect of the candidate chemotherapeutic agent on the cancer cell relative to a reference cytotoxic effect, wherein an increase in cytotoxic effect relative to the reference cytotoxic effect indicates that the candidate chemotherapeutic agent is a wild-type bacterium responsive chemotherapeutic agent. In one embodiment, the method of the invention identifies a combination of chemotherapeutic drugs all of which respond to a specific wild-type bacteria.

Thus, for any specific wild-type bacterium, it is possible to identify chemotherapeutic agents that are responsive to the specific wild-type bacterium. It is also possible to identify wild-type bacteria that activate specific chemotherapeutic agents (hereafter “chemotherapeutic activating wild-type bacteria” or “chemotherapeutic activating wild-type bacterium”).

The invention also provides a method for treating cancer in a mammal comprising administering to the mammal a synergistic combination comprising an effective dose of a chemotherapeutic-activating wild-type bacterium and an effective dose of at least one chemotherapeutic agent that is responsive to the chemotherapeutic activating wild-type bacterium (hereafter “wild-type bacterium-responsive chemotherapeutic agent”). In one embodiment, the combination comprises 2, 3, 4, 5, 6, 7, 8, or 9 wild-type bacterium responsive chemotherapeutic agents.

The invention also provides a pharmaceutical composition comprising a chemotherapeutic activating wild-type bacterium, at least one wild-type bacterium-responsive chemotherapeutic agent, and a pharmaceutically acceptable excipient. In one embodiment, the composition comprises 2, 3, 4, 5, 6, 7, 8, or 9 wild-type bacterium responsive chemotherapeutic agents.

The invention also provides a chemotherapeutic-activating wild-type bacterium in combination with at least one chemotherapeutic agent that is responsive to the chemotherapeutic activating wild-type bacterium, for use in the prevention or treatment of cancer. In one embodiment, the therapeutic use employs 2, 3, 4, 5, 6, 7, 8, or 9 wild-type bacterium responsive chemotherapeutic agents.

The invention also relates to a method of treating or preventing a cancer in an individual comprising the steps of assay a tumour sample from the individual for the presence of a wild-type bacterium, wherein when the presence of a wild-type bacterium is detected, administering to the individual at least one chemotherapeutic agent that is responsive to the chemotherapeutic activating wild-type bacterium In one embodiment, 2, 3, 4, 5, 6, 7, 8, or 9 wild-type bacterium responsive chemotherapeutic agents are administered.

In another aspect, the invention provides a method for identifying a chemotherapeutic agent that is inhibited by a specific wild-type bacterium (hereafter “wild-type bacterium susceptible chemotherapeutic agent”) comprising the steps of incubating the specific wild-type bacterium with a candidate chemotherapeutic agent for a period of time, separating the candidate chemotherapeutic agent from the wild-type bacterium, incubating the candidate chemotherapeutic agent with a cancer cell, and determining whether there is an decrease in a cytotoxic effect of the candidate chemotherapeutic agent on the cancer cell relative to a reference cytotoxic effect, wherein a decrease in cytotoxic effect relative to the reference cytotoxic effect indicates that the candidate chemotherapeutic agent is a wild-type bacterium susceptible chemotherapeutic agent.

Thus, for any specific wild-type bacterium, it is possible to identify chemotherapeutic agents that are inhibited by the specific wild-type bacterium, and therefore formulate or prescribe therapies accordingly, for example providing a cancer therapy comprising a wild-type bacterium susceptible chemotherapeutic agent bacterium and an anti-bacterial agent effective against the wild-type bacterium.

The invention therefore also provides for a method for preventing or treating cancer in a mammal comprising administering to the mammal a combination therapy comprising an effective dose of a wild-type bacterium susceptible chemotherapeutic agent and an effective dose of at least one anti-bacterial agent effective against the wild-type bacterium. The anti-bacterial agent may be specific to the wild-type bacterium, or a broad-spectrum anti-bacterial agent that is effective against the wild-type bacterium.

The invention also provides a pharmaceutical composition comprising a wild-type bacterium susceptible chemotherapeutic agent, at least one anti-bacterial agent effective against the wild-type bacterium, and a pharmaceutically acceptable excipient.

The invention also provides for a wild-type bacterium susceptible chemotherapeutic agent and at least one anti-bacterial agent effective against the wild-type bacterium, for use in the prevention or treatment of cancer characterised by the presence of the wild-type bacterium.

The invention also relates to a method of identifying a suitable chemotherapeutic agent for a mammal having a tumour comprising the steps of assaying the tumour for the presence of at least one chemotherapeutic activating wild-type bacterium, wherein when the presence in the tumour of the at least one chemotherapeutic activating wild-type bacterium is confirmed, identifying a wild-type bacterium-responsive chemotherapeutic agent as a suitable chemotherapeutic agent for the mammal and optionally treating the mammal with the identified wild-type bacterium-responsive chemotherapeutic agent.

The invention also relates to a method for the prevention or treatment of cancer in an individual comprising the steps of identifying a suitable chemotherapeutic agent for the mammal according to a method of the invention, and then administering an effective dose of the identified chemotherapeutic agent to the mammal.

The invention also relates to a method of treating or preventing a cancer in an individual comprising the steps of assay a tumour sample from the individual for the presence of a chemotherapeutic inhibiting wild-type bacteria, wherein when the presence of chemotherapeutic-inhibiting wild-type bacteria is detected, administering to the individual an anti-bacterial agent directed against the chemotherapeutic-inhibiting wild-type bacteria and a wild-type bacteria non-responsive chemotherapeutic agent.

The invention also relates to a method of biotransforming a chemotherapeutic agent comprising the step of incubating the chemotherapeutic agent with a chemotherapeutic-activating wild-type bacteria, or an extract of the chemotherapeutic-activating wild-type bacteria (for example, a cell lysis extract), for a period of time sufficient to allow the chemotherapeutic-activating wild-type bacteria or extract thereof convert the chemotherapeutic agent to a biotransformed chemotherapeutic agent, and then optionally separating the biotransformed chemotherapeutic agent from the bacteria or extract thereof.

In one embodiment, the bacterium is a probiotic bacterium.

In one embodiment, the chemotherapeutic agent is a prodrug. In one embodiment, the chemotherapeutic agent is an enzyme-modifiable chemotherapeutic drug.

In one embodiment, the composition comprises (or the therapeutic method/use comprises administration of) an enzyme-modifiable chemotherapeutic drug and a bacterium that expresses an enzyme that activates the enzyme-modifiable chemotherapeutic drug. In one embodiment, the composition comprises (or the therapeutic method/use comprises administration of) a plurality of enzyme-modifiable chemotherapeutic drugs and a bacteria that expresses an enzyme that activates the plurality of enzyme-modifiable chemotherapeutic drugs. In one embodiment, the bacterium is selected from E. coli, Listeria, Lactobacillus, Bifidobacteria, and Lactococcus. and the chemotherapeutic prodrug is optionally one or more of CB1954, 5-FC, AQ4N and Fludarabine Phosphate. In one embodiment, the bacteria is selected from E. coli Nissei, Listeria welshimeri, Lactobacillus salivarius, Bifidobacteria breve, and Lactococcus lactis. and the chemotherapeutic prodrug is optionally two, three or all of CB1954, 5-FC, AQ4N and Fludarabine Phosphate. In one embodiment, the E. coli is E. coli Nissle.

### BRIEF DESCRIPTION OF THE DRAWINGS

The patent or application file contains at least one drawing executed in color. Copies of this patent or patent application publication with color drawings will be provided by the Office upon request and payment of the necessary fee.

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIGS. 1A-1D: Tumour Cell Survival

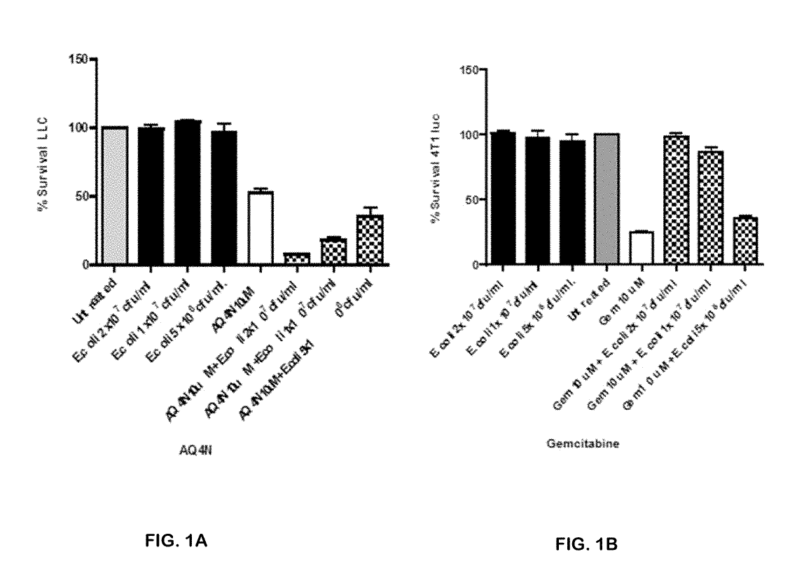

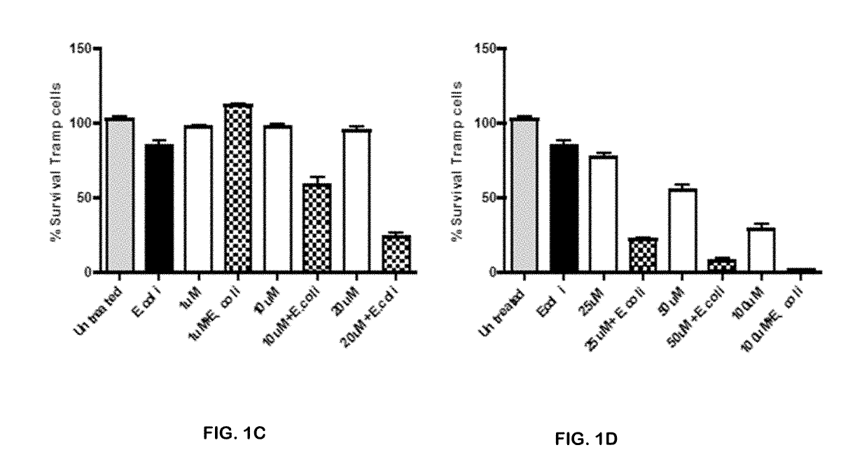

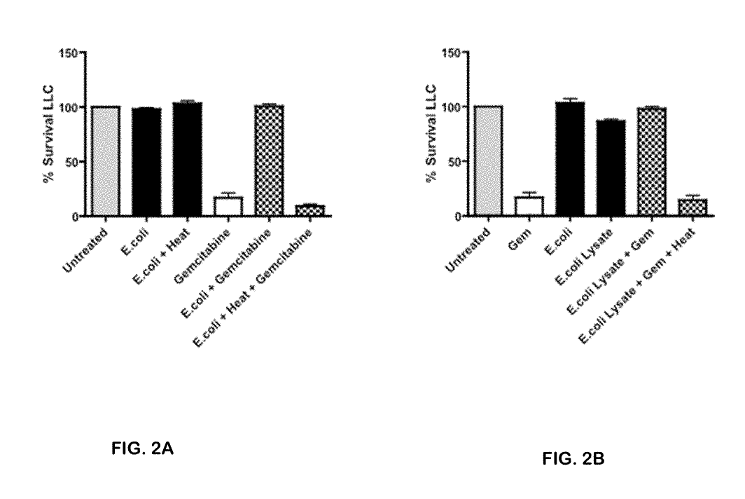

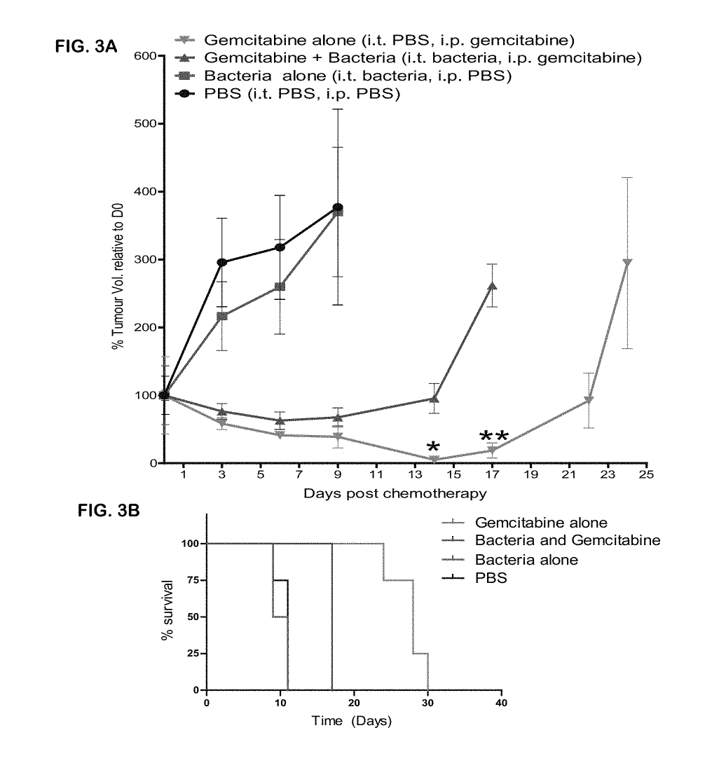

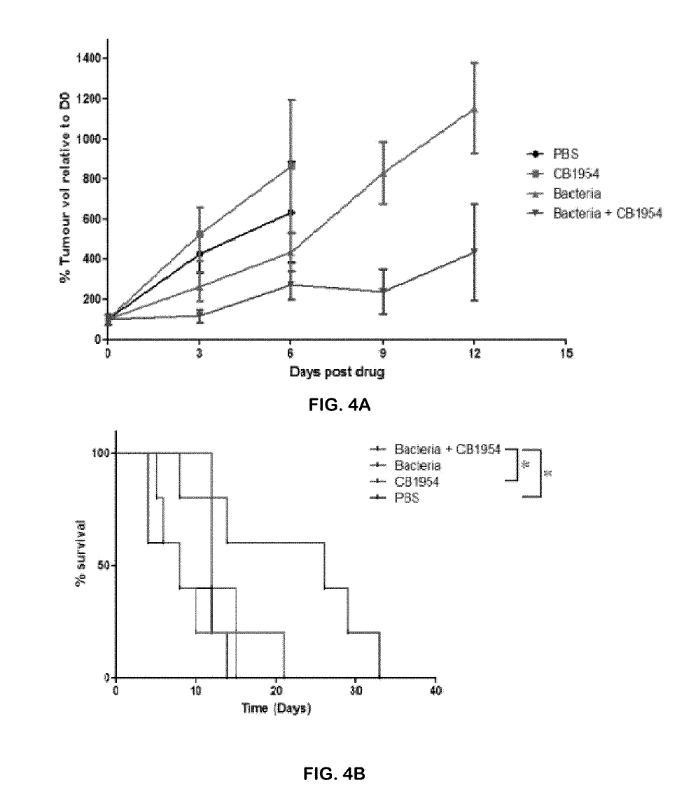

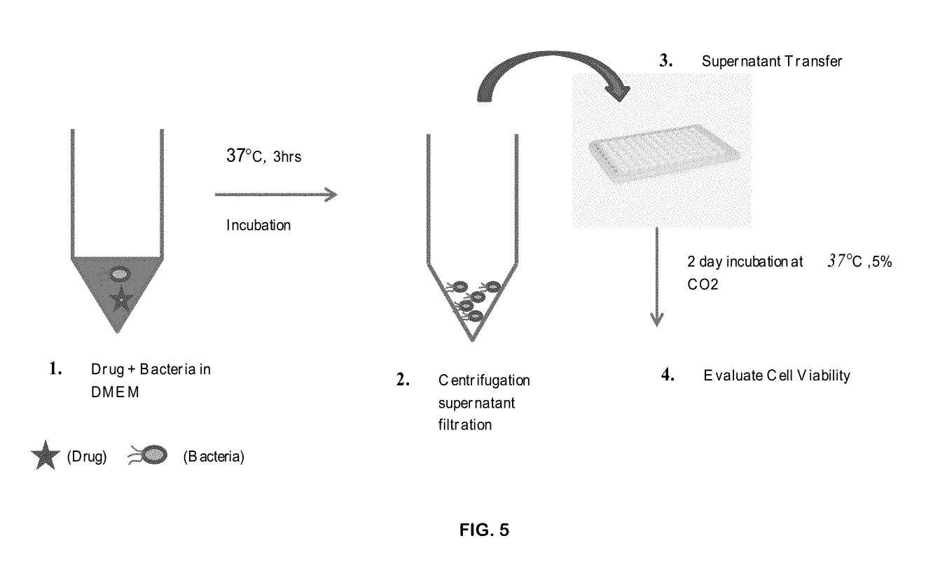

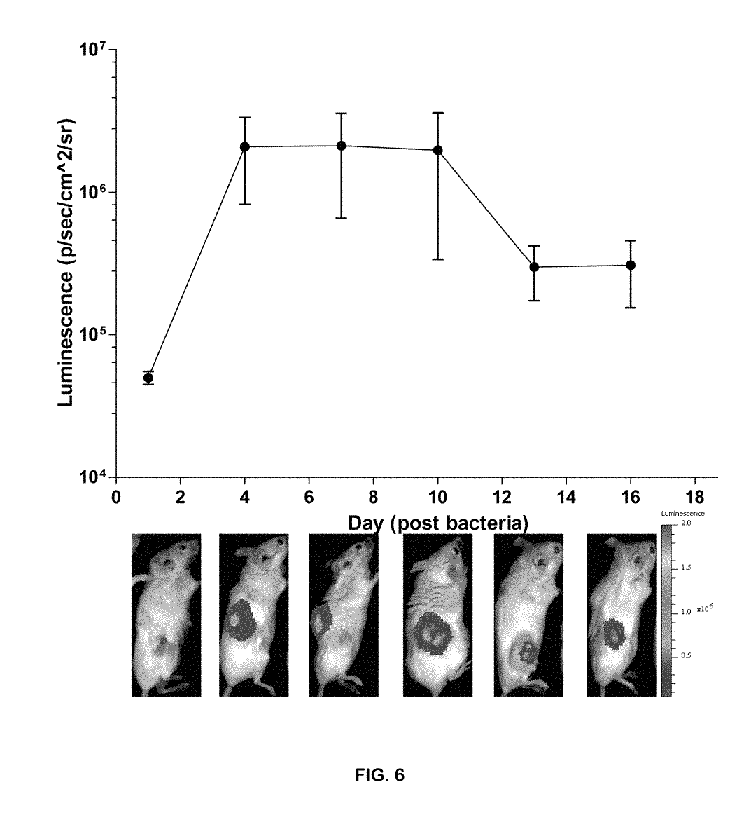

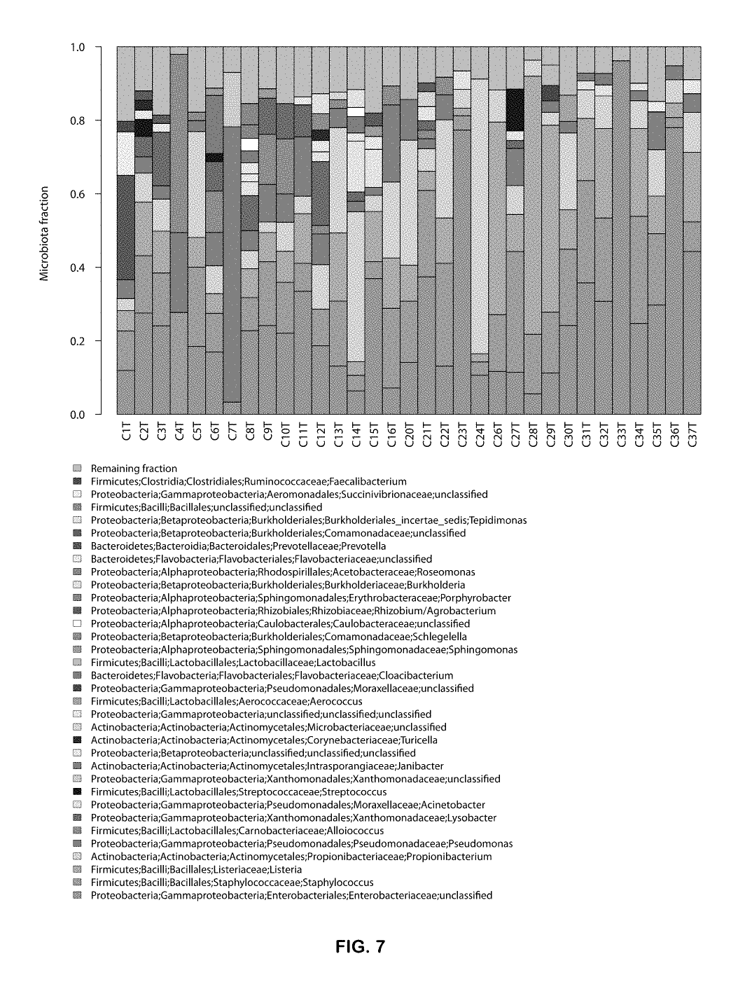

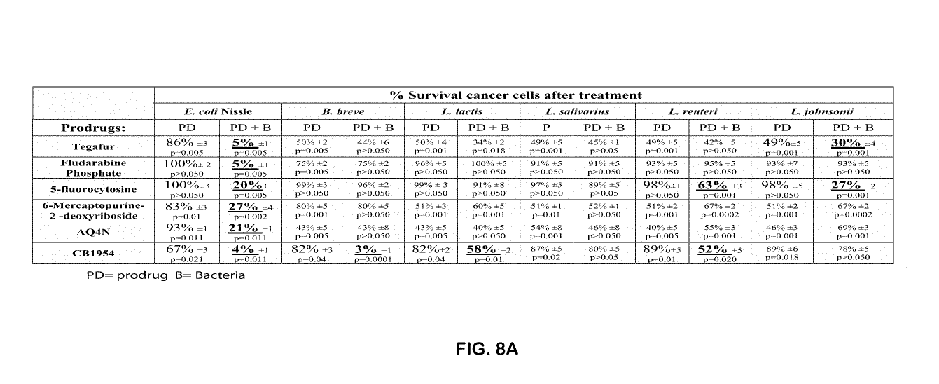

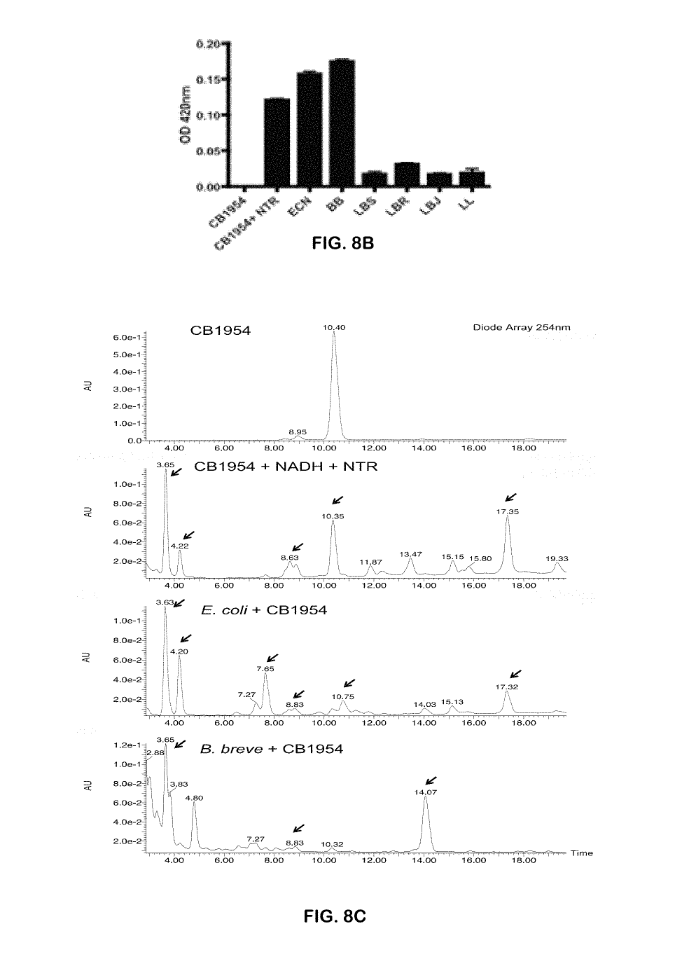

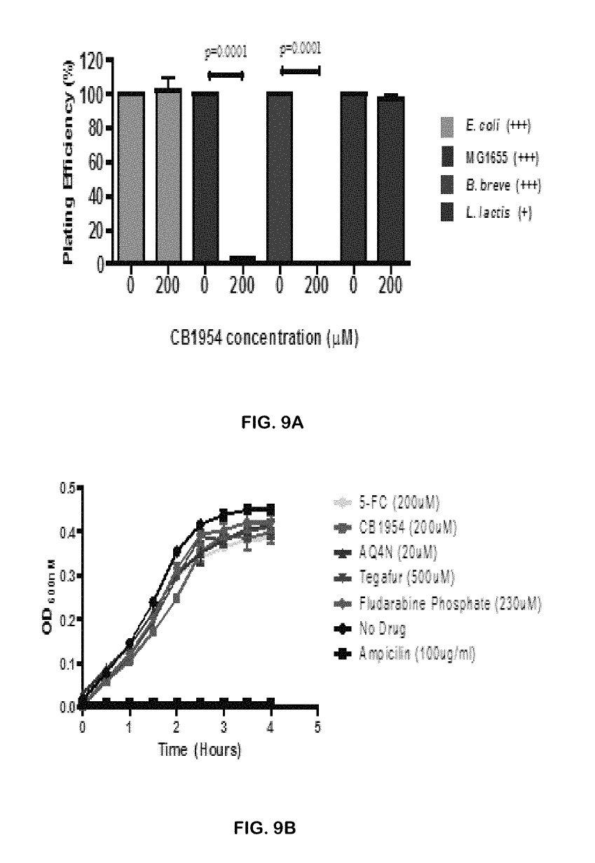

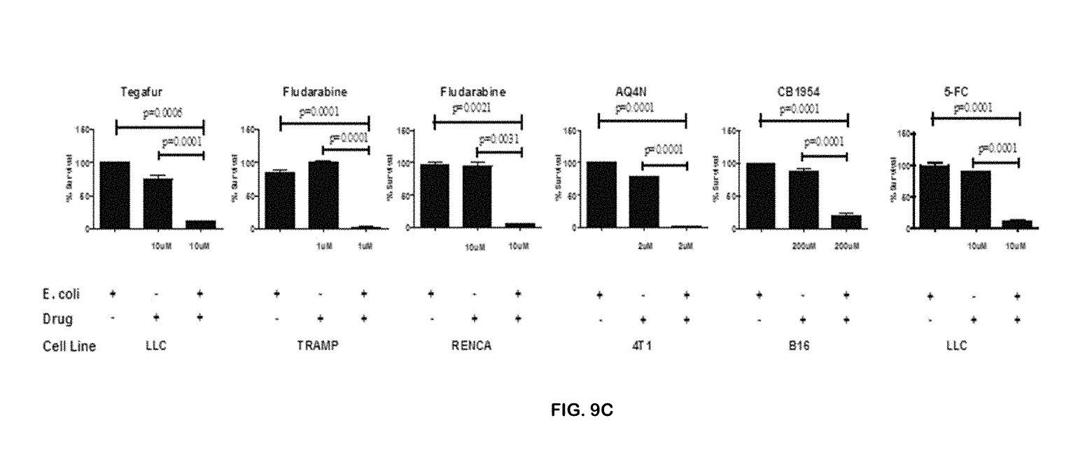

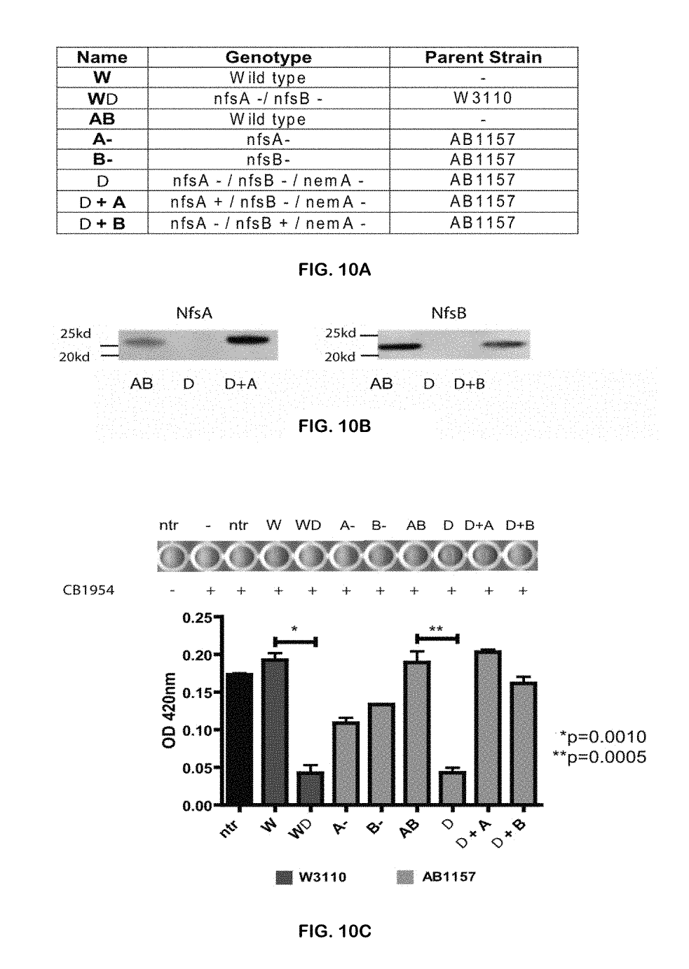

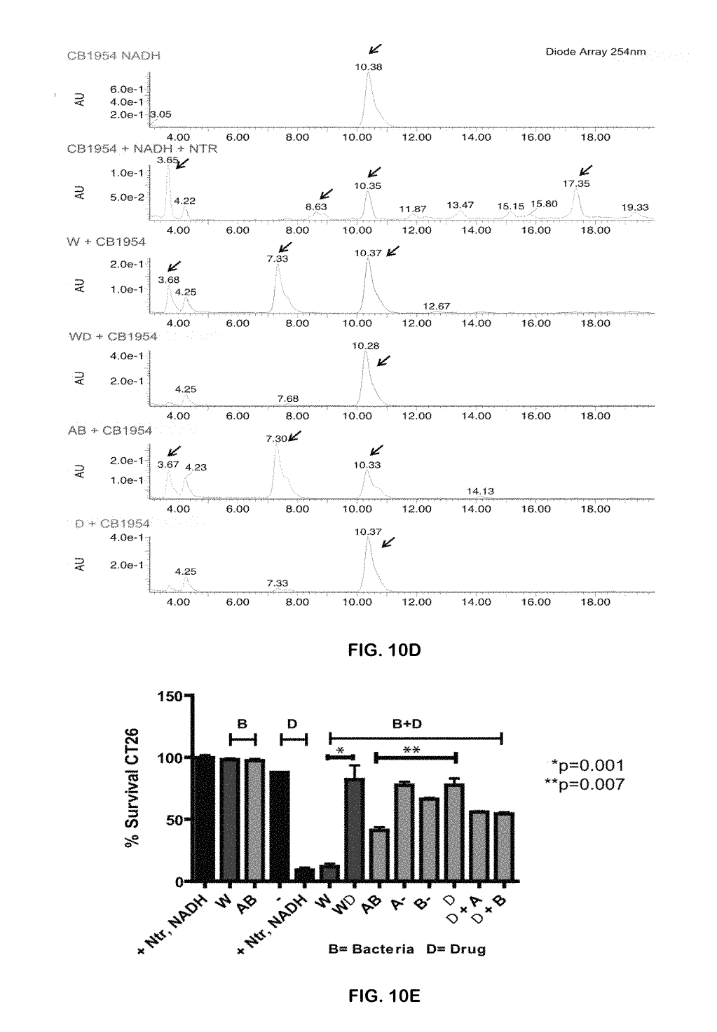

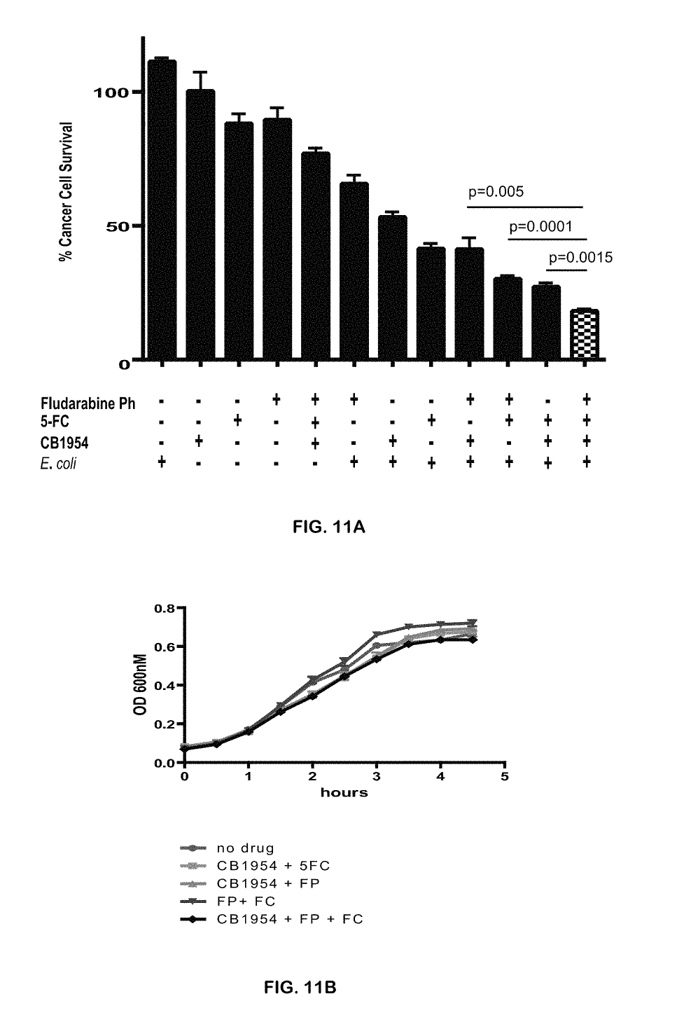

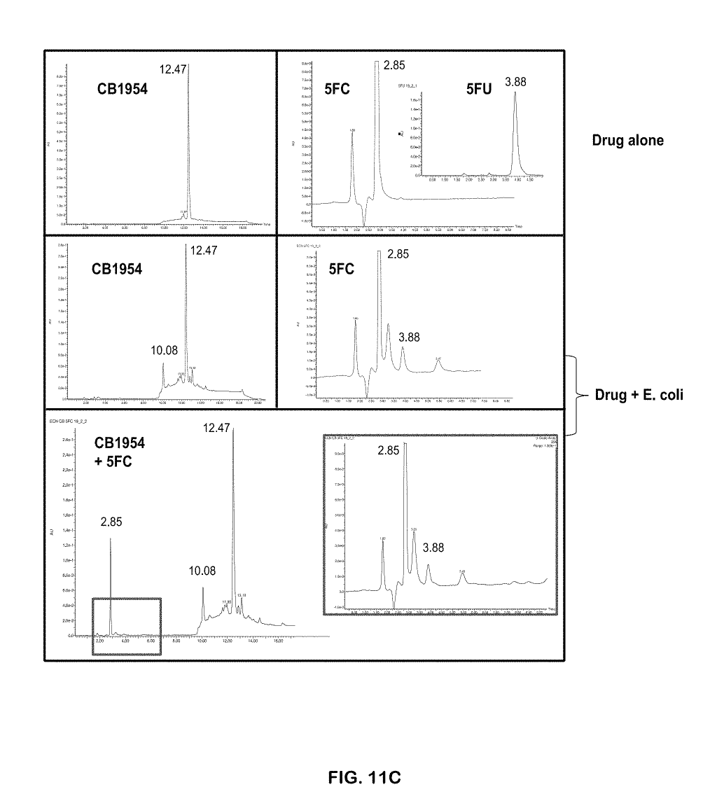

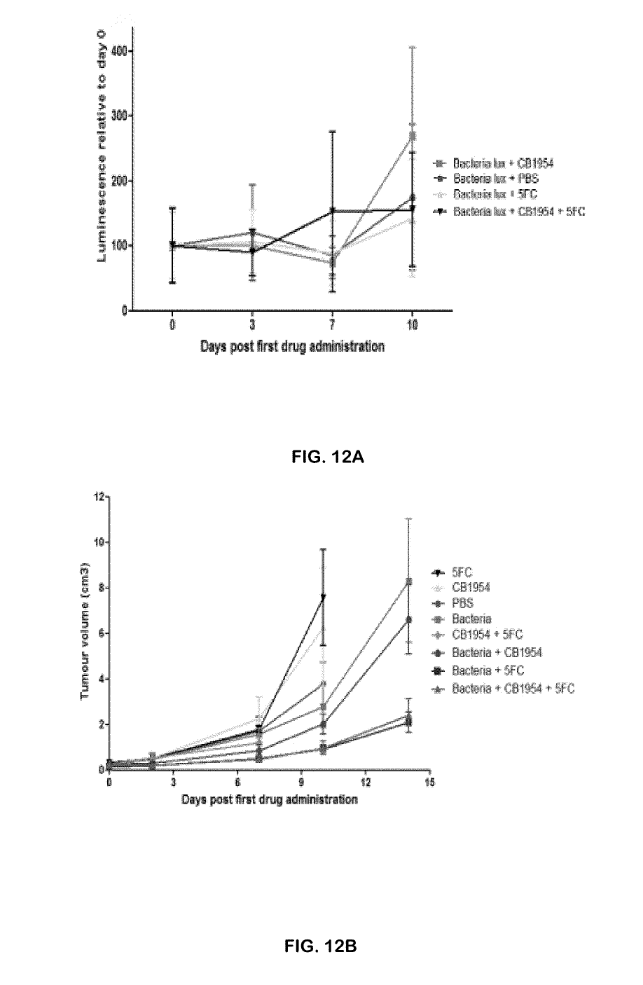

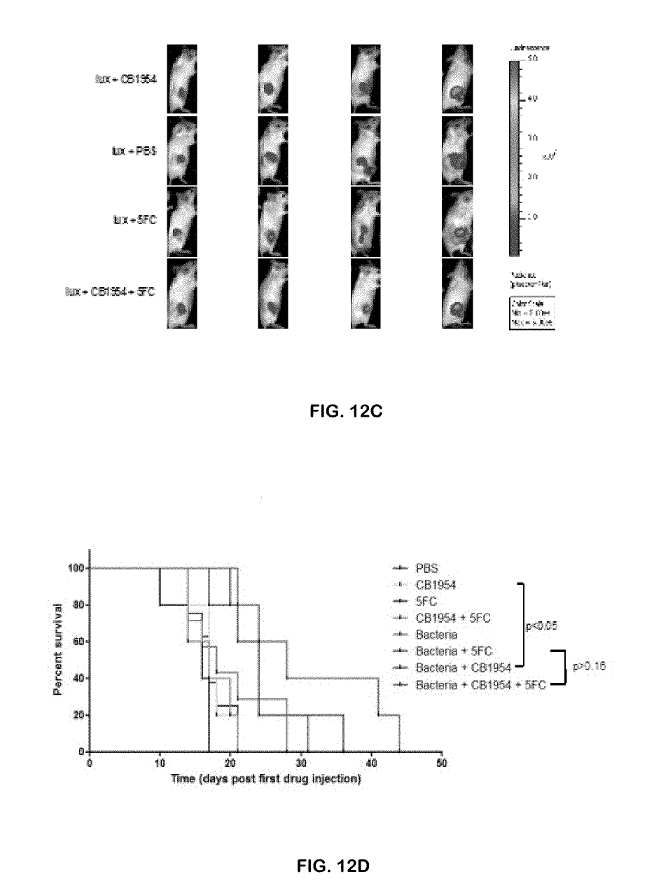

Cell survival assay. See FIG. 5 for assay schematic. FIG. 1A) E. coli at different cfu/ml were co-incubated with AQ4N (10 μM) after which the supernatant was applied directly to LLC cells (P<0.01). FIG. 1B) E. coli at different cfu/ml were co-incubated with gemcitabine (10 μM) after which the supernatant was directly applied to 4T1 Luc cells (P<0.01). FIG. 1C)-FIG. 1D) E. coli was co-incubated with Tegafur (C)) or CB1954 (D)) at the indicated concentrations after which the supernatant was directly applied to TRAMP cells (P<0.01). Data represent the average and standard error of three technical replicates.

FIGS. 2A-2B: Cell Lysate Assays

FIG. 2A) Tumour cell survival assay stained with MTS. Gemcitabine (10 μM) was incubated with live or heat killed E. coli (P<0.001). FIG. 2B) Tumour cell survival assay. Gemcitabine (10 μM) was incubated with either bacterial lysate (equivalent amounts to cell survival assay live bacteria dosages) alone or bacterial lysate that has been heat inactivated (P<0.001). Data represent the average and standard error of three technical replicates.

FIGS. 3A-3B: E. coli Decreases the Efficacy of Gemcitabine In Vivo

Subcutaneous flank CT26 tumours growing in Balb/c mice were injected i.t with bacteria or PBS vehicle alone. Gemcitabine (60 mg/kg) was injected i.p. five times at three day intervals. FIG. 3A) Tumour volume (%) relative to the first day of gemcitabine injection (day 0) is shown. *P<0.03, **P=0.002 for gemcitabine alone versus gemcitabine+bacteria. FIG. 3B) The median survival post Day 0 of the gemcitabine+bacteria group was significantly less than that of the gemcitabine alone group (17 days vs. 28 days +/−1.25; P=0.008). Data are expressed as mean±SEM of 4 to 8 individual mice per group.

FIG. 4A-4B: E. coli Increases the Cytotoxicity of CB1954

Subcutaneous flank CT26 tumours growing in Balb/c mice were injected i.t with bacteria or PBS vehicle alone. CB1954 (20 mg/kg) was injected i.p. for the duration of the experiment at 3 day intervals. FIG. 4A) Tumour volume (%) relative to the first day of CB1954 injection (day 0) is shown. FIG. 4B) The median survival post Day 0 of the CB1954 +bacteria group was significantly greater than that of the CB1954 alone group (26 days vs. 8 days. P=0.0374). Data are expressed as mean±SEM of 3-5 individual mice per group.

FIG. 5: Tumour Survival Assay

1. Bacteria and Drug are mixed in DMEM medium, and co-incubated for 3 h at 37° C. 2. The drug is separated from bacteria using centrifugation and filtration. 3. The filter sterilised DMEM containing the transformed drug is applied to tumour cells in a 96 well plate. 4. Tumour cell viability is evaluated via the MTS assay for end point measurements.

FIG. 6: Growth of i.t. Administered Bacteria in Mouse Tumours

1×10⁶ lux-expressing E. coli was i.t. administered to s.c. CT26 flank tumours growing in Balb/c mice. Viable, luminescent bacteria were detected at various time points specifically in the tumour using whole body imaging. A graphical representation of tumour luminescence over time plotted as the mean +/−SEM for Four mice, with a representative mouse image under corresponding time points.

FIG. 7: Analysis of Tumour Microbiota

FIGS. 8A-8C: Live Wild Type Bacteria Induce Cytotoxicity by Activating Prodrugs.

FIG. 8A) Prodrug conversion by probiotic strains. The data presented were generated in the tumour cell line CT26. Drug efficacy is represented by percentage survival of cancer cells after treatment (490 nm reading of treated samples/490 nm reading of untreated sample×100). Highlighted in bold are bacteria-prodrug combinations that induced statistically significant toxicity relative to drug alone. Each set of data (PD/PD+B) are representative independent experiments (n>3). SEM is represented by ±. FIG. 8B) Absorbance readings of supernatants of live bacteria-CB1954 (200 μM) incubations measured at 420 nm. Control: purified NfsB protein (10 μg), NADH (1 mM), CB1954 (200 μM) at 37° C., blanked with CB1954 (n=4) C) HPLC chromatograms of bacterial supernatants and solutions as indicated; CB1954 (300 μM).

FIGS. 9A-9C: In vitro evaluation of E. coli as a therapeutic agent in conjunction with prodrugs. FIG. 9A) Bacterial survival in the presence of CB1954. Bacterial cells in logarithmic phase were plated directly on to LB plates containing CB1954 (200 μM) and grown overnight. The plating efficiency shown is normalised to cfu counts from plain LB plates (+:prodrug efficacy on cancer cells). FIG. 9B) Prodrug influence on bacterial growth. Prodrugs were incubated with bacteria in LB medium and the change in optical density at 600 nm was monitored over time at 37° C. Data represent the average and standard error of four technical replicates. These data are representative of 2 independent experiments. FIG. 9C) Therapeutic efficacy of E. coli in conjunction with prodrugs on multiple cell lines. Survival was measured by the MTS assay and is expressed relative to untreated cells. Data represent the average and standard error of four technical replicates. These data are representative of 2 independent experiments.

FIGS. 10A-10E: Different enzymes can contribute to prodrug activation. FIG. 10A) Table of the different strains used in this study. The abbreviations, the genotype and strain name they were derived from are listed. FIG. 10B) Nfs protein expression characterisation using Western blot with anti-nfs A or B antibodies. AB parent strain, the AB triple nitroreductase knockout mutant and the latter reconstituted with either nitroreductase A or B are shown. FIG. 10C) CB1954 conversion by live bacteria, indicated by a change of colour (top) and change of absorbance at 420 nm over time (bottom). The derivative strains are grouped by colour indicating their parent strains. FIG. 10D) HPLC chromatogram of drug, NADH and live bacteria co-incubations. A positive control containing CB1954, purified nfsB protein (10 μg/ml) and NADH (1 mM) is included (second row). FIG. 10E) Cancer survival assay following co-incubation of CB1954 (100 μM) and E. coli variants. Data represent the average and standard error of four technical replicates. These data are representative of 2 independent experiments.

FIGS. 11A-11C: Multiple prodrugs can be co-activated by E. coli. FIG. 11A) Cancer cell survival in the presence of multiple prodrugs. Bacteria were incubated with treated prodrugs for 1 h. Fludarabine phosphate (5 μM), 5-FC (2 μM), CB1954 (25 μM) FIG. 11B) Prodrug influence on bacterial growth. Prodrugs were incubated with bacteria in LB medium and the change in optical density at 600 nm was monitored over time at 37° C. (CB1954: 200 μM, 5-FC: 500 μM, FP (Fludarabine phosphate): 20 μM). FIG. 11C) HPLC chromatogram of supernatants of E. coli previously co-incubated with prodrugs in PBS at 37° C. Top; drugs alone (purified 5-FU is run as a control) middle: Single drug-bacterial incubations. Bottom; Cocktail incubations. Red rectangle: Magnification of 5-FC profile in this reaction.

FIGS. 12A-12D: Tumour therapy via in vivo activation of prodrugs. BALB/c mice bearing subcutaneous CT26 flank tumours colonised by E. coli Nissle 1917 received IP injections of CB1954+PBS, 5-FC+PBS, CB1954+5-FC or PBS three times per week. Tumours were monitored for changes in volume and luminescence (where applicable) from live bacteria on each treatment day. FIG. 12A) The change in bacterial luminescence (relative to day 0) from tumour colonising luminescent E. coli is shown. Luminescence remains stable across the range of time-points indicating that bacterial levels within the tumour remained constant throughout the experiment. FIG. 12B) Changes in CT26 tumour volume were monitored until the first death in the group determined by a predefined endpoint. FIG. 12C-12D) Survival plot of each group starting from the first day of bacteria/drug administration. Mice that received bacteria and both drugs showed significantly increased median survival (p<0.01) compared with groups that did not receive bacteria.

### DETAILED DESCRIPTION OF THE INVENTION

A description of example embodiments of the invention follows.

Definitions:

“Cancer”: means the group of diseases involving abnormal cell growth with the potential to spread to distant or local sites, especially means a malignant tumour. In one embodiment, the cancer is a solid tumour. Typically, the cancer is selected from the group comprising: esophagogastric cancer; fibrosarcoma; myxosarcoma; liposarcoma; chondrosarcoma; osteogenic sarcoma; chordoma; angiosarcoma; endotheliosarcoma; lymphangiosarcoma; lymphangioendotheliosarcoma; synovioma; mesothelioma; Ewing's tumor; leiomyosarcoma; rhabdomyosarcoma; colon carcinoma; pancreatic cancer; breast cancer; ovarian cancer; prostate cancer; squamous cell carcinoma; basal cell carcinoma; adenocarcinoma; sweat gland carcinoma; sebaceous gland carcinoma; papillary carcinoma; papillary adenocarcinomas; cystadenocarcinoma; medullary carcinoma; bronchogenic carcinoma; renal cell carcinoma; hepatoma; bile duct carcinoma; choriocarcinoma; seminoma; embryonal carcinoma; Wilms' tumor; cervical cancer; uterine cancer; testicular tumor; lung carcinoma; small cell lung carcinoma; bladder carcinoma; epithelial carcinoma; glioma; astrocytoma; medulloblastoma; craniopharyngioma; ependymoma; pinealoma; hemangioblastoma; acoustic neuroma; oligodendroglioma; meningioma; melanoma; retinoblastoma; and leukemias.

“Solid tumour” is a cancer that is not a haematological malignancy. Two of the main types of solid tumours are sarcomas and carcinomas. Sarcomas are tumours in a blood vessel, bone, fat tissue, ligament, lymph vessel, muscle or tendon. They include Ewing sarcoma and Osteosarcoma, which are bone cancer sarcomas, and Rhabdomyosarcoma, which is a soft tissue carcinoma found in muscles. Carcinomas are tumours that form in epithelial cells. Epithelial cells are found in the skin, glands and linings of the organs, including the bladder, ureters, and part of the kidneys.

“Treatment”: means a course of action/dosing regime that either inhibits, delays or prevents the progression of cancer, including cancer metastasis, or that inhibits, delays or prevents the recurrence of cancer, including cancer metastasis, or that prevents or hinders the onset or development of cancer in an individual.

“Prevention”: means prevention of the occurrence or recurrence of cancer, at a local or distant site, typically following the withdrawal of chemotherapeutic drugs in an individual diagnosed with cancer.

“Chemotherapeutic agent”: means an agent that induces cancerous cells to commit to cell death. Suitable chemotherapeutic agents will be known to those skilled in the art. Such chemotherapeutic agents include but are not limited to; alkylating agents, anti-metabolites, plant alkyloids and terpenoids, topoisomerase inhibitors, anti-tumour antibiotics and platinum based compound including but not limited to cisplatin, carboplatin and oxaliplatin. Examples of suitable chemotherapeutic anti-metabolites include, purine analogues not limited to azathoprine, mercaptopurine, tioguanine and fludarabine; pyrimidine analogues not limited to 5-fluorouracil (5-FU), floxuridine and cytosine arabinoside; antifolates not limited to methotrexate, trimethoprim, pyrimethamine and pemetrexed. Suitably, the chemotherapeutic agent is an anti-metabolite. In one embodiment, the chemotherapeutic is a prodrug (i.e. designed to be activated at a tumour site). In one embodiment, the chemotherapeutic is an prodrug that is enzyme activated (enzyme modifiable chemotherapeutic drug) and capable of being employed in enzyme directed chemotherapeutic prodrug therapy. Enzyme modifiable chemotherapeutic prodrug therapy is a broad term used to describe two-step strategies which aim to localise chemotherapeutic activity to the site of the tumour, therefore limiting adverse side effects and permitting administration of higher drug doses. Traditionally, a gene coding for an enzyme that has the ability to convert a non-toxic prodrug to a toxic drug is used to arm a cancer gene therapy vector (e.g. ligand [2], antibody [3], virus [4], bacterium [5]). Firstly, the vector is administered to the patient in order to deliver the gene to the tumour cells. There, the gene becomes expressed and sensitises the cells to a specific prodrug. In the second step, the relevant prodrug is administered to the patient and is converted to a toxic drug specifically by enzyme-expressing tumour cells resulting in localized toxicity while sparing distant healthy tissue. Specific examples of enzyme modifiable chemotherapeutic drugs are described in the literature ([6], [7]) and include Tagefur, Fludarabine Phosphate, 5-fluorocysteine, 6-mercaptupurine-2-deoxyriboside, AQ4N and CB1954.

“Wild-type bacteria” or “wild-type bacterium”: means naturally occurring bacteria that are not genetically modified. Preferably, the term means wild-type bacteria known to inhabit mammalian tumours, especially malignant mammalian tumours, (hereafter “mammalian tumor resident wild-type bacterium”) examples of which are described in the literature and FIG. 7. In one embodiment of the invention the wild-type bacteria is a species of the taxa/genera E. coli, typically E.coli nissle. In another embodiment the wild-type bacteria is a species of the taxa/genera Listeria, typically Listeria welshimeri. In another embodiment the wild-type bacteria is a species of the taxa/genera Enterobacteriacae. In another embodiment the wild-type bacteria is a species of the taxa/genera Staphylococcus. In another embodiment the wild-type bacteria is a species of the taxa/genera Propionibacterium. In another embodiment the wild-type bacteria is a species of the taxa/genera Pseudomonas. In another embodiment the wild-type bacteria is a species of the taxa/genera Alloiococcus. In another embodiment the wild-type bacteria is a species of the taxa/genera lysobacter. In another embodiment the wild-type bacteria is a species of the taxa/genera Acinetobacter. In another embodiment the wild-type bacteria is a species of the taxa/genera Streptococcus. In another embodiment the wild-type bacteria is a species of the taxa/genera Xanthomonadaceae. In another embodiment the wild-type bacteria is a species of the taxa/genera Janibacter. In another embodiment the wild-type bacteria is a species of the taxa/genera Betaproteobacteria. In another embodiment the wild-type bacteria is a species of the taxa/genera Turicella. In another embodiment the wild-type bacteria is a species of the taxa/genera Microbacteriaceae. In another embodiment the wild-type bacteria is a species of the taxa/genera Gammaproteobacteria. In another embodiment the wild-type bacteria is a species of the taxa/genera Aerococcus. In another embodiment the wild-type bacteria is a species of the taxa/genera Moraxellaceae. In another embodiment the wild-type bacteria is a species of the taxa/genera Cloacibacterium. In another embodiment the wild-type bacteria is a species of the taxa/genera Lactobacillus. In another embodiment the wild-type bacteria is a species of the taxa/genera Sphingomonas. In another embodiment the wild-type bacteria is a species of the taxa/genera Scxhlegella. In another embodiment the wild-type bacteria is a species of the taxa/genera Caulobacteraceae. In another embodiment the wild-type bacteria is a species of the taxa/genera Rhizobiiaceae. In another embodiment the wild-type bacteria is a species of the taxa/genera Porphyrobacter. In another embodiment the wild-type bacteria is a species of the taxa/genera Burkholderia. In another embodiment the wild-type bacteria is a species of the taxa/genera Roseomonas. In another embodiment the wild-type bacteria is a species of the taxa/genera Flavobacteriaceae. In another embodiment the wild-type bacteria is a species of the taxa/genera Prevotella. In another embodiment the wild-type bacteria is a species of the taxa/genera Comamonadacae. In another embodiment the wild-type bacteria is a species of the taxa/genera Tepidomonas. In another embodiment the wild-type bacteria is a species of the taxa/genera Bacillales. In another embodiment the wild-type bacteria is a species of the taxa/genera Succinivivrionaceae. In another embodiment the wild-type bacteria is a species of the taxa/genera Faecalibacterium. In another embodiment the wild-type bacteria is a species of the taxa/genera Bifidobacteria. In another embodiment the wild-type bacteria is a species of the taxa/genera Lactococcus.

In one embodiment, the wild-type bacteria is a probiotic bacteria. In one embodiment, the bacteria is a gram positive bacteria. In one embodiment, the bacteria is a gram negative bacteria. In one embodiment, the wild-type bacteria is selected from E.coli, Listeria, Lactobacillus, Bifidobacteria, and Lactococcus. In one embodiment, the wild-type bacteria is selected from E. coli Nissei, Listeria welshimeri, Lactobacillus salivarius, Bifidobacteria breve, and Lactococcus lactis.

“Probiotic” as applied to a bacteria means a bacteria that when consumed by a human confers health benefits on the human. Examples of probiotic strains of bacteria include Lactobacillus acidophilus Rosen-52, Lactobacillus rhamnosis gg, and Bifidobacterium infantis Rosen-53. Probiotic bacteria are described in [8].

“Cytotoxic effect”: means the inhibition of cell growth or induction of cell death.

“Reference cytotoxic effect”: means the cytotoxic effect of a chemotherapeutic agent against a cancer cell carried out in the absence of a wild-type bacteria, or where the chemotherapeutic agent is not pre-incubated with the wild-type bacteria.

“Synergistic combination”: means a combination of actives that is used in cancer therapy and comprises a wild-type bacteria known to activate (i.e. increase the cytotoxic effect) of a specific chemotherapeutic agent and a chemotherapeutic agent known to be activated by the wild-type bacteria. The actives in the synergistic combination may be administered together (co-administration) or separately, for example at different times of the day or on different days. Preferably, the bacteria is administered to the patient prior to the drug, typically at least 1, 3, 5, 7, 9, 11, 13, 15, 17 or 19 days prior to administration of the chemotherapeutic agent. The synergistic combination may be provided in a form for co-administration of the actives (i.e. in the form of a unit dose product containing both actives) or for sequential or separate administration of the actives (i.e. a kit comprising the two actives provided separately, for example two unit dose products).

“Wild-type bacteria-responsive chemotherapeutic agent” or “chemotherapeutic agent that is responsive to the chemotherapeutic activating wild-type bacteria”: means a chemotherapeutic agent that is activated by the wild-type bacteria (i.e. exhibits an increased cytotoxic effect or is converted to a more cytotoxic form in the presence of the bacteria). Without being bound by theory, it is believed that the chemotherapeutic agent is biotransformed by one or more specific wild-type bacteria to a more cytotoxic chemotherapeutic form. Examples of wild-type bacteria-responsive chemotherapeutic agent are provided below and include Tegafur, Fludarabine de phosphate, 5-fluorocysteine, 6-mercaptopurine-2′-deoxyriboside, and AQ4N which are responsive to E. coli, L. welshimeri, or both.

“Chemotherapeutic-activating wild type bacterium”: means wild-type bacterium that are capable of increasing the cytotoxic effect of one or more chemotherapeutic agents. Examples of chemotherapeutic-activating wild type bacteria include E. coli Nissei, Listeria welshimeri, Lactobacillus salivarius, Bifidobacteria breve, and Lactococcus lactis.

“Chemotherapeutic-inhibiting wild type bacterium”: means wild-type bacterium that are capable of decreasing the cytotoxic effect of one or more chemotherapeutic agents. Examples of chemotherapeutic-inhibiting wild type bacteria include E. coli and L. welshimeri.

“Effective dose”: as applied to a wild-type bacterium means a dosage of the bacteria that is sufficient to increase the cytotoxic effect of the wild-type bacteria responsive chemotherapeutic agent in vivo. In one embodiment, an effective dose is at least 1×10⁷, 1×10⁸ or 1×10⁹ bacteria per millilitre in a suitable carrier.

“Biotransformed chemotherapeutic agent”: means a chemotherapeutic agent that is biotransformed by a chemotherapeutic-activating wild type bacterium to more cytotoxic form.

“Effective dose”: as applied to a chemotherapeutic agent means an amount of a chemotherapeutic agent which results in partial or total inhibition in the progression of cancer and/or prevents or inhibits the recurrence of cancer following withdrawal from an anti-cancer regime. In a particular, a therapeutically effective amount of a chemotherapeutic agent should be taken to mean an amount that results in a clinically significant number of cancer cells being killed. An effective amount can be readily determined by the attending diagnostician, as one skilled in the art, by the use of known techniques and by observing results obtained under analogous circumstances. In determining the effective amount or dose of compound administered, a number of factors are considered by the attending diagnostician, including, but not limited to: the type of chemotherapeutic agent; species of mammal; its size, age, and general health; the specific disease involved; the degree of or involvement or the severity of the disease; the response of the individual patient; the particular compound administered; the mode of administration; the bioavailabilty characteristics of the preparation administered; the dose regimen selected; the use of concomitant medication; and other relevant circumstances. As an example, the following doses may be employed:

Cisplatin: high dose=6.9 mg/kg; low dose=2 mg/kg

Lithium Chloride: high dose=14.5/17 mg/kg; low dose=4.5/10 mg/kg

Rapamycin: high dose=2 mg/kg; low dose=0.6 mg/kg

5-Fluorouracil: high dose=87 mg/kg; low dose=8/12 mg/kg

In this specification, the term “administering” should be taken to include any form of delivery that is capable of delivering the chemotherapeutic agent and the wild-type bacteria (or anti-bacterial agent) to cancer cells including intravenous delivery, oral delivery, intramuscular delivery, intrathecal delivery, transdermal delivery, inhaled delivery and topical delivery. Methods for achieving these means of delivery will be well known to those skilled in the art of drug delivery. The term should also encompass co-administration of the two actives, or administration at separate times. For example, the actives may be administered on alternate days, or on the same day at different times, or on different days of the week. Preferably, the bacteria is administered to the patient prior to the drug, typically at least 1, 3, 5, 7, 9, 11, 13, 15, 17 or 19 days prior to administration of the chemotherapeutic agent.

“Anti-bacterial agent” means an agent that can kill or prevent the growth of bacteria (i.e. bacteriocidal or bacteriostatic). The agent may be a narrow spectrum or broad spectrum agent, meaning that it is effective against a small range of bacteria or effective against a broad range of bacteria. A detailed list of antibacterial agents can be found at www.chem.msu.su/rus/books/patrick/part2.pdf. Such an agent may be administered with a chemotherapeutic drug such as Cladribine which is shown herein to have reduced cytotoxic effect in the presence of E. coli and L. welshimeri.

Experimental

Materials and Methods:

Bacteria, mammalian cell lines and drugs

E. coli Nissle 1917 (UCC culture collection) was cultivated aerobically in L-Broth or L-Agar (Sigma) at 37° C. Bioluminescent E. coli was described by us previously [9] and cultured in the presence of 300 mg/ml erythromycin. Listeria welshimeri Serovar 6B SLCC5334 was purchased from ECACC and cultivated at 37° C. in Brain Heart Infusion (BHI) medium. B. breve UCC2003 and Lactococcus lactis (UCC Culture Collection) was routinely grown at 37° C. in reinforced clostridial medium and M17 medium respectively (Oxoid, Basingstoke, UK). All lactobacilli strains were a kind gift of Dr. Cormac Gahan, UCC School of microbiology, and were grown in lactobacillus MRS broth (Beckton Dickinson). The E. coli nitroreductase deletion mutants and parent strains were kindly provided by Dr. Antonio Valle [10] and grown in LB broth/agar with appropriate antibiotics. The strain Δ+A was created by transforming AB502 (streptomycin, kanamycin sensitive derivative of parent strain AB1157) with the plasmid pNZ44 (chloramphenicol resistance) carrying the gene nfsA. The strain Δ+B was created by transforming AB502 with the plasmid pTrc99A (ampicillin resistance) containing the gene nfsB (see below).

Cancer cell lines were purchased from ATCC and were propagated according to the supplier's instructions. The murine recycled prostate cancer cell line TRAMPC1 was kindly provided by Ciavarra RP [11] of Eastern Virginia Medical School, Norfolk USA, and propagated accordingly [12].

AQ4N was purchased from TOCRIS bioscience (UK). The other prodrugs, NADH, and Nitroreductase B (NfsB) were purchased from Sigma (Ireland). Chemicals were resuspended in DMSO or water according to their supplier's specifications.

Drugs

All drugs and enzymes were purchased from Sigma except: Etoposide Phosphate (Santa Cruz), Capecitabine (Santa Cruz), AQ4N (R&D), Nelarabine (A&B), and Vidarabine (Santa Cruz). Drugs were resuspended in H₂O or DMSO, with appropriate control vehicle utilised accordingly in all experiments.

Cloning of nfsB in AB502

Standard molecular biology techniques were employed. Briefly; PCR primers incorporating restriction sites NcoI and XbaI were designed for amplification of the nfsB gene from genomic DNA of MG1655. The KOD polymerase kit (Novagen) was used for the amplification of nfsB gene and the PCR reaction was performed according to the manufacturer's instructions. The nfsB fragment was gel purified, digested with NcoI and XbaI restriction enzymes and cloned into pTrc99A to yield pTrc99A-nfsB which was subsequently used to transform electrocompetent AB502 cells.

Cell Cytotoxicity Assay

Microtitre plates (96-well) were pre-seeded with 4000 cells/well in appropriate medium for each cell line and allowed to attach overnight. The next day, bacteria were cultured to log-phase or stationery phase (Gram-positive) resuspended in PBS and added to 2 mls of DMEM to an OD₆₀₀ₘₙ of 0.1-0.2 for E. coli 0.05 for Lactococcus lactis, and 0.3-0.4 for the rest of the Grand-positives. Each strain was exposed for 2-3 h to drug in a tissue culture incubator. Bacteria were separated from the drug by centrifugation and filter sterilisation using 0.2 μm pore filters (Starsted) after which 200 μl of filtrate was transferred to each well. Plates were incubated until cells in control (untreated) had achieved confluent growth (FIG. S1). Cytotoxicity was quantified using an MTS staining with the Cell Titre 96 AQueous One solution Cell Proliferation Assay (Promega). The plates were measured and analysed in a Spectra max M2 (Molecular devices) spectrophotometer at 490 nm.

Bacterial Lysis and Heat Inactivation

Bacteria were heat inactivated at 95° C. for 40 min. Lysis was facilitated by sonication using three 10 sec pulses (at 20 Kz, 50 W). Between each pulse, samples were incubated on ice for 30 seconds. A 20-fold drop in optical density was considered sufficient lysis.

Detection of NfsA and NfsB by Western Blotting

For protein extraction, bacteria grown to log phase were pelleted and frozen at −70° C. overnight. The pellets were resuspended in lysis buffer (25 mM Tris-Cl, 2 mM EDTA, 15 mg.mL⁻¹ lysozyme) at 37° C. for 1 h and briefly sonicated on ice. Protein concentration of total cell lysates was determined using a Bradford Assay standard curve. Total cell lysate protein (60 μg) was separated using the Novex NuPAGE SDS-PAGE Gel System (Invitrogen) and electroblotted onto PDVF membranes. The latter were blocked using Odyssey blocking buffer (Li-Cor) and probed with a polyclonal anti-NfsA or anti-NfsB antibody (kindly provided by Dr. Peter Searle, Birmingham, UK) both diluted 1:1000. Immune complexes were detected by fluorescence by means of an odyssey infrared scanner and the band intensity quantified with the associated software.

CB1954 Colour Assay

CB1954 products produce a yellow colour which correlates with an increase in absorbance at 420 nm. Overnight bacterial cultures were washed in PBS and incubated with 300 μM CB1954 for 3 h at 37° C. Bacteria were pelleted and 200 μL from each supernatant (in quadruplicate) were plated onto a 96-well plate and measured at 420 nm in a Spectra max M2 (Molecular devices) spectrophotometer. NfsB (10 μg) was used in reactions which contained 0.2% DMSO and 1 mM NADH and were either supplemented with or without CB1954.

Bacterial Plating Assay

Bacteria were grown to log phase and plated directly on to agar plates containing the appropriate growth medium with 200 μM CB1954 or without. Colonies were counted and scored after an overnight incubation at 37° C. Survival of bacteria in the presence of CB1954 was expressed in relation to untreated controls.

Bacterial Prodrug Susceptibility Assay

Overnight bacterial cultures were used to inoculate fresh LB medium +/−prodrugs and transferred into 96 well format (300 μl/well) for automated tracking in a plate reader. The change in optical density at 600 nm (37° C.) of each individual well was monitored over several hours (in a Spectra max M2 device) in the presence of drugs.

HPLC and Mass Spectrometry Analyses

Sample preparation: Bacteria were grown overnight (5 mls) in appropriate media and environment. The bacteria were harvested washed once in PBS and then resuspended in the same volume of PBS. 1 ml/5 ml was removed and used for each assay. Bacteria and drug were incubated for 2 h at 37° C., and centrifuged at 13,500 rpm for 5 min. Supernatant containing drug was analysed by HPLC.

Method CB1954 gradient: The results described within were obtained using a Waters Micromass LCT Premier mass spectrometer (Instrument number KD160). Analysis was performed in ESI+mode using a gradient elution method to identify unknowns in the sample. An external reference standard of Leucine enkephalin was infused in order to confirm mass accuracy of the MS data acquired. The samples were run in triplicate to ensure consistency and the data was analysed by used of Masslynx 4.1 software. LC conditions: a Waters Alliance 2695 with a 2996 Photodiode Array detector and Waters Atlantis T3 C18 5 □m 150×4.6 mm HPLC column was used for the chromatographic separation with mobile phase: Acetonitrile (containing 0.1% formic acid) and Water (containing 0.1% formic acid) using the following gradient: 0 min (10:90); 0.5 min (10:90); 17 min (30:70); 17.5 min (10:90); 20 min (10:90). A flow rate of 1 ml/min, sample run time of 20 min and injection volume of 100 μL was used. The MS conditions were as follows: the samples were subjected to ESI+ionisation and acquired from 80 to 1250 m/z at a capillary voltage of 3.00 kV, sample cone of 30 V and a source temperature of 140° C. An external calibration was applied using Sodium Formate solution and an external reference solution of Leucine Enkephalin in Water/Acetonitrile (ESI+m/z=556.2771) for exact mass correction using Lockspray was used. The UV conditions were set at a sampling rate of 1 spectrum/second, scanning wavelengths from 195-500 nm at a resolution of 1.2 nm.

Method CB1954/5-FC gradient: The results described within were obtained using a Waters Micromass LCT Premier mass spectrometer (Instrument number KD160). Analysis was performed in ESI+and ESI-mode using a gradient elution method to identify unknowns in the sample. An external reference standard of Leucine enkephalin was infused in order to confirm mass accuracy of the MS data acquired. The samples were run in triplicate to ensure consistency and the data was analysed by used of Masslynx 4.1 software. LC conditions: a Waters Alliance 2695 with a 2996 Photodiode Array detector and Waters Atlantis T3 C18 □m 150×4.6 mm HPLC column was used for the chromatographic separation with mobile phase: Acetonitrile (containing 0.1% formic acid) and Water (containing 0.1% formic acid) using the following gradient: 0 min (0:100); 0.5 min (0:100); 6 min (0:100); 14 min (80:20); 16 min (80:20); 16.5 min (0:100); 20 min (0:100). A flow rate of 1 ml/min, sample run time of 20 min and injection volume of 100 μL was used. The MS conditions were as follows: the samples were subjected to ESI+ (or ESI−) ionisation and acquired from 80 to 1250 m/z at a capillary voltage of 3.00 kV, sample cone of 30 V and a source temperature of 140° C. An external calibration was applied using Sodium Formate solution and an external reference solution of Leucine Enkephalin in Water/Acetonitrile (ESI+m/z=556.2771; ESI−m/z=554.2615) for exact mass correction using Lockspray was used. The UV conditions were set at a sampling rate of 1 spectrum/second, scanning wavelengths from 195-500 nm at a resolution of 1.2 nm.

Murine Experiments

Animals and tumour induction: 6-8 week old female BALB/c mice weighing approximately 18-20 g were kept as previously described. For tumour induction, a 200 μl suspension of 2×10⁵ CT26 cells in serum free Dulbecco's Modified Eagle's Medium (DMEM) culture medium were injected subcutaneously into the flank of BALB/c mice. The viability of inoculated cells was determined using the Nucleocounter system (ChemoMetec). Tumour growth was monitored three times per week by caliper measurement and mice were randomly assigned to groups when the tumours reached approximately 100 mm³ in volume. Mice were culled once tumours reached 1.5×1.5 cm in size (predetermined endpoint).

Bacterial administration to mice: Bacteria were grown overnight in LB medium and in a shaking incubator at 37° C. These bacteria were used to inoculate fresh growth medium and allowed to grow until reaching OD₆₀₀ 0.6. Cultures were harvested by centrifugation (4000×g for 10 min) and washed three times in phosphate buffered saline (PBS) before resuspension in one-tenth volume PBS. For tumour colonisation, approximately 5×10⁵ bacteria were injected directly into the tumour at 3 locations. The total volume of bacteria injected was 50 μl. Mice that did not receive bacteria were injected with an equal volume of PBS.

Drug administration to mice: Mice were treated with drug+PBS or both drugs three times per week. The concentration and volume of drugs used were as follows; CB1954 (20 mg/kg in 100 μl PBS+DMSO), 5-FC (200 mg/kg in 300 μl PBS+DMSO) or PBS. Each drug/PBS injection was separate so all mice received two intraperitoneal injections on treatment days to a total volume of 500 μl.

Image acquisition and formation:In vivo bioluminescence imaging was carried out using the IVIS Lumina II (Perkin Elmer). Mice were imaged for bioluminescence on the same day as drug treatment and regions of interest were quantified using LivingImage 4.3.1 software. Representative images of mice show a randomly selected mouse from each group (minimum of n=3 in all cases).

Ethics statement: All murine experiments were approved by the animal ethics committee of University College Cork (AERR #2010/003 and #2012/015). Mice were monitored for signs of illness throughout the course of experiments. No adverse events were recorded.

Statistical analysis For biological in vitro and in vivo assays, two-sided, paired student's t test with 95% confidence or Mann Whitney U test were employed to investigate statistical differences, using GraphPad Prism@5.0 or Microsoft Excel 12. Multiple comparison tests were carried out using the Bonferroni post hoc test. Statistical significance of survival between groups in murine experiments was determined using the Log-rank (Mantel-Cox) Test. Statistical significance was defined at the 5% level. Survival curves are presented as Kaplan-Meier plots.

TABLE 1
Summary of observations from in vitro cytotoxicity screen of drugs with
bacteria using the bacterial cell kill assay

| | | Cytotoxicity | |
|---|---|---|---|
| | | E. | L. |
| Drug Name | Drug Class | coli | welshimeri |
| Tegafur | Anti-metabolite | Up | NC |
| Fludarabine de | Anti-metabolite | Up | Up |
| phosphate | | | |
| Capecitabine | Anti-metabolite | NC | NC |
| 5-fluorocytosine | Anti-metabolite | Up | n/d |
| 5-fluorouracil | Anti-metabolite | NC | NC |
| 6-Mercaptopurine- | Anti-metabolite | Up | n/d |
| 2′-deoxyriboside | | | |
| Pentostatin | Anti-metabolite | NC | NC |
| Cytarabine | Anti-metabolite | NC | NC |
| Clofarabine | Anti-metabolite | NC | NC |
| Cladribine | Anti-metabolite | Down | Down |
| Valacyclovir | Anti-metabolite | NC | n/d |
| Ara G hydrate | Anti-metabolite | NC | NC |
| Nelarabine | Anti-metabolite | NC | NC |
| Vidarabine | Anti-metabolite | Down | NC |
| Gemcitabine | Anti-metabolite | Down | NC |
| Doxorubicin | Anti-tumour Antibiotics | Down | n/d |
| Daunorubicin | Anti-tumour Antibiotics | Down | Down |
| Vinblastine | Anti-tumour Antibiotics | NC | n/d |
| Actinomycin | Anti-tumour Antibiotics | NC | n/d |
| Idarubicin | Anti-tumour Antibiotics | Down | n/d |
| Mitomycin C | Anti-tumour Antibiotics | NC | n/d |
| Streptonegrin | Anti-tumour Antibiotics | NC | n/d |
| Etoposide phosphate | Topo Isomerase Inhibitor | Down | NC |
| Irinotecan | Topo Isomerase Inhibitor | NC | NC |
| AQ4N | Topo Isomerase Inhibitor | Up | n/d |
| Mitoxantrone | Topo Isomerase Inhibitor | Down | n/d |
| B-Lapachone | Topo Isomerase Inhibitor | Down | n/d |
| Estramustine | Alkylating Agent | NC | n/d |
| CB1954 | Alkylating Agent | Up | Up |
| Menadione | Reactive oxygen generator | Down | n/d |

### REFERENCES

[1]. S. Viaud et al, Science, Vol. 342, No. 6161, November 2013.

[2] D. Morrissey, G. C. O'Sullivan, M. Tangney, Tumour targeting with systemically administered bacteria, Curr Gene Ther, 10 (2010) 3-14.

[3] C. K. Baban, M. Cronin, D. O'Hanlon, G. C. O'Sullivan, M. Tangney, Bacteria as vectors for gene therapy of cancer, Bioeng Bugs, 1 (2010) 385-394.

[4] M. Zhao, M. Yang, X. M. Li, P. Jiang, E. Baranov, S. Li, M. Xu, S. Penman, R. M. Hoffman, Tumor-targeting bacterial therapy with amino acid auxotrophs of GFP-expressing Salmonella typhimurium, Proc Natl Acad Sci U S A, 102 (2005) 755-760.

[5] J. Stritzker, P. J. Hill, I. Gentschev, A. A. Szalay, Myristoylation negative msbB-mutants of probiotic E. coli Nissle 1917 retain tumor specific colonization properties but show less side effects in immunocompetent mice, Bioeng Bugs, 1 (2010) 139-145.

[6] Connors et al. Stem Cells 1995 September; 13(5).

[7] Rooseboom et al, Pharmacological Reviews; 2004 March; 56(1).

[8]. McFarland LV. From yaks to yogurt: the history, development, and current use of probiotics. Clinical infectious diseases: an official publication of the Infectious Diseases Society of America 2015; 60 Suppl 2: S85-90

[9] M. Cronin, A. R. Akin, S. A. Collins, J. Meganck, J. B. Kim, C. K. Baban, S. A. Joyce, G. M. van Dam, N. Zhang, D. van Sinderen, G. C. O'Sullivan, N. Kasahara, C. G. Gahan, K. P. Francis, M. Tangney, High resolution in vivo bioluminescent imaging for the study of bacterial tumour targeting, PLoS One, 7 (2012) e30940.

[10] A. Valle, S. Le Borgne, J. Bolivar, G. Cabrera, D. Cantero, Study of the role played by NfsA, NfsB nitroreductase and NemA flavin reductase from Escherichia coli in the conversion of ethyl 2-(2′-nitrophenoxy)acetate to 4-hydroxy-(2H)-1,4-benzoxazin-3(4H)-one (D-DIBOA), a benzohydroxamic acid with interesting biological properties, Applied microbiology and biotechnology, 94 163-171.

[11] K. D. Somers, R. R. Brown, D. A. Holterman, N. Yousefieh, W. F. Glass, G. L. Wright, Jr., P. F. Schellhammer, J. Qian, R. P. Ciavarra, Orthotopic treatment model of prostate cancer and metastasis in the immunocompetent mouse: efficacy of flt3 ligand immunotherapy, Int J Cancer, 107 (2003) 773-780.

[12] S. Ahmad, G. Casey, P. Sweeney, M. Tangney, G. C. O'Sullivan, Prostate stem cell antigen DNA vaccination breaks tolerance to self-antigen and inhibits prostate cancer growth, Mol Ther, 17 (2009) 1101-1108.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. A method of treating a solid tumour cancer in a mammal comprising the step of administering to the mammal an effective dose of a chemotherapeutic-activating wild-type bacterium and an effective dose of a chemotherapeutic agent that is responsive to the chemotherapeutic activating wild-type bacterium.

2. A method according to claim 1 in which the bacterium and chemotherapeutic agent are administered to the mammal intravenously.

3. A method according to claim 1 in which the chemotherapeutic-activating wild-type bacterium is a probiotic bacterium.

4. A method according to claim 1 in which the chemotherapeutic agent is a chemotherapeutic prodrug.

5. A method according to claim 1 in which the chemotherapeutic-activating wild-type bacterium is selected from E.coli, Listeria, Lactobacillus, Bifidobacteria, and Lactococcus.

6. A method according to claim 1 in which the chemotherapeutic agent is selected from Tegafur, Fludarabine de phosphate, 5-fluorcytosine, 6-mercaptopurine-2′-deoxyriboside, CB1954 and AQ4N.

1. d according to claim 1 in which the chemotherapeutic-activating wild-type bacterium is selected from E. coli, Listeria, Lactobacillus, Bifidobacteria, and Lactococcus, and in which the chemotherapeutic agent is selected from Tegafur, Fludarabine de phosphate, 5-fluorcytosine, 6-mercaptopurine-2′-deoxyriboside, CB1954 and AQ4N.

8. A composition comprising a chemotherapeutic-activating wild-type bacterium and a chemotherapeutic agent that is responsive to the chemotherapeutic activating wild-type bacterium.

9. A composition according to claim 8 formulated for intravenous administration.

10. A composition according to claim 8 in which the chemotherapeutic agent is an enzyme-modifiable chemotherapeutic drug.

11. A composition according to claim 8 wherein the chemotherapeutic-activating wild-type bacterium is selected from E. coli, Listeria, Lactobacillus, Bifidobacteria, and Lactococcus.

12. A composition according to claim 8, wherein the chemotherapeutic agent is selected from Tegafur, Fludarabine de phosphate, 5-fluorcytosine, 6-mercaptopurine-2′-deoxyriboside, CB1954 and AQ4N.

13. A composition as claimed in claim 8 in which the chemotherapeutic-activating wild-type bacterium is selected from E. coli, Listeria, Lactobacillus, Bifidobacteria, and Lactococcus, and in which the chemotherapeutic agent is selected from Tegafur, Fludarabine de phosphate, 5-fluorcytosine, 6-mercaptopurine-2′-deoxyriboside, CB1954 and AQ4N.

14. A method of treating a solid tumour cancer in a mammal comprising the steps of administering to the mammal an effective dose of a wild-type bacterium susceptible chemotherapeutic agent and an effective dose of an anti-bacterial agent effective against the wild-type bacterium.

15. A method according to claim 14 in which the anti-bacterial agent and chemotherapeutic agent are administered to the mammal systemically.

16. A method according to claim 14 in which the chemotherapeutic-activating wild-type bacterium is selected from E. coli, Listeria, Lactobacillus, Bifidobacteria, and Lactococcus.

17. A method according to claim 14 in which the wild-type bacterium susceptible chemotherapeutic agent is selected from Cladribine, Vidarabine, Gemcitabine, Doxorubicin, Daunorubicin, Idarubicin, Etoposide phosphate, Mitoxantrone, B-Lapachone, and Medadione.
